(19) United States
(12) Patent Application Publication
(10) Pub. No.: US 2013/0017999 A1
(43) Pub. Date: Jan. 17, 2013

(54) Methods and Compositions for Evaluating and/or Treating Chronic Immune Diseases

(71) Applicants: Marc Fremont, Zellik (BE); Kenny Leo De Meirleir, Mechelen (BE)

(72) Inventors: Marc Fremont, Zellik (BE); Kenny Leo De Meirleir, Mechelen (BE)

(21) Appl. No.: 13/549,352

(22) Filed: Jul. 13, 2012

Related U.S. Application Data
(60) Provisional application No. 61/507,970, filed on Jul. 14, 2011.
(60) Provisional application No. 61/570,702, filed on Dec. 14, 2011.

(51) Int. Cl. C40B 20/00; A61K 31/711; A61K 31/713; A61K 38/00; A61P 25/00

(52) U.S. Cl. 514/17.9; 506/2; 514/17.7; 514/44 A; 514/44 R

(57) ABSTRACT

Methods and compositions are provided for evaluating a subject for chronic immune disease, including predicting whether a subject is susceptible to a chronic immune disease, diagnosing whether a subject has a chronic immune disease and/or determining a treatment for a subject suffering from a chronic immune disease. Aspects of the methods include obtaining an intestinal bacterial assessment for the subject and using the assessment to provide the evaluation. In addition, reagents and kits thereof that find use in practicing the subject methods are provided. Furthermore, methods of treating a subject for a chronic immune disease are provided.

### CROSS REFERENCE To RELATED APPLICATIONS

Pursuant to 35 U.S.C. §119 (e), this application claims priority to the filing date of the U.S. Provisional Patent Application Ser. No. 61/507,970 filed Jul. 14, 2011 and United States Provisional Patent Application Ser. No. 61/570,702 filed Dec. 14, 2011; the disclosures of which are herein incorporated by reference.

### INTRODUCTION

Chronic Fatigue Syndrome (CFS)/myalgic encephalomyelitis is defined by a severe and debilitating fatigue associated with a variety of symptoms including musculoskeletal pain, sore throat, tender lymph nodes, sleep abnormalities, and neurocognitive problems (Fukuda et al. (1994) Ann Intern Med 121:953-959). The pathogenesis of CFS is still poorly understood, but is likely to be multifactorial; viral infections, stress, neuroendocrine dysfunctions, exposure to toxins have all been proposed as contributing factors to the onset and maintenance of the disease (Devanur and Kerr (2006) J Clin Virol 37(3):139-50).

### SUMMARY

Methods and compositions are provided for evaluating a subject for chronic immune disease, including predicting whether a subject is susceptible to a chronic immune disease, diagnosing whether a subject has a chronic immune disease and/or determining a treatment for a subject suffering from a chronic immune disease. Aspects of the methods include obtaining an intestinal bacterial assessment for the subject and using the assessment to provide the evaluation. In addition, reagents and kits thereof that find use in practicing the subject methods are provided. Furthermore, methods of treating a subject for a chronic immune disease are provided.

### DETAILED DESCRIPTION

Methods and compositions are provided for evaluating a subject for chronic immune disease, including predicting whether a subject is susceptible to a chronic immune disease, diagnosing whether a subject has a chronic immune disease and/or determining a treatment for a subject suffering from a chronic immune disease. Aspects of the methods include obtaining an intestinal bacterial assessment for the subject and using the assessment to provide the evaluation. In addition, reagents and kits thereof that find use in practicing the subject methods are provided. Furthermore, methods of treating a subject for a chronic immune disease are provided.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, aspects of the invention are directed to methods of evaluating chronic immune disease including predicting whether a subject is susceptible to a chronic immune disease, diagnosing whether a subject has a chronic immune disease or determining a treatment for a subject suffering from a chronic immune disease, as well as reagents and kits thereof (and devices) for use in practicing the subject methods.

### Methods of Evaluating a Subject for Chronic Immune Disease

Embodiments of the invention are directed to evaluating a subject for a chronic immune disease, such as Chronic Fatigue Syndrome. By evaluating is meant making some assessment or determination about a subject and a chronic immune disease of interest. As an example, aspects of the methods include: (a) predicting whether a subject is susceptible to a chronic immune disease; (b) diagnosing a subject as having a chronic immune disease; and/or (c) determining a treatment in a subject suffering from a chronic immune disease. Examples of chronic immune diseases are Chronic Fatigue Syndrome and Multiple Sclerosis. In further describing aspects of the invention, the following description focuses on Chronic Fatigue Syndrome. However, the subject methods and the reagents, devices and kits thereof also find use in the prediction of susceptibility to, diagnosis of, and treatment of other chronic immune diseases as well, as described above.

Chronic Fatigue Syndrome (CFS), also known as myalgic encephalomyelitis (ME), is a condition in which the subject suffers from fatigue associated with symptoms which may include but are not limited to muscle and joint (musculoskeletal) pain, sore throat, tender lymph nodes, sleep abnormalities, and/or neurocognitive problems. Often, the symptoms last for six months or more. Often, the fatigue is debilitating. In certain embodiments, the methods are directed at CFS and particularly at whether a subject will or will not be susceptible to CFS, at diagnosing a subject as having CFS, or at determining what treatment to provide a subject with CFS.

Aspects of the invention include methods of predicting whether a subject is or is not susceptible to CFS, or is or is not resistant to CFS. By predicting is meant making a forecast or prognostication as to whether a subject may suffer from CFS at a future date. In some instances subject is susceptible to CFS if the subject is more likely to develop CFS than an individual of the general population, whereas a subject that is resistant to CFS is less likely to develop CFS than an individual of the general population. The predication may not be 100% accurate.

Additional aspects of the invention include methods of diagnosing whether a subject suspected of having CFS has CFS. Subjects suspected of having CFS and thus amenable to these methods can be identified using any convenient protocol. One convenient protocol is diagnosis based on clinical symptoms. A number of different clinical symptoms may be used to identify subjects that may have or have CFS. For example, clinical symptoms of interest include: fatigue of six months or longer that causes a reduction in effort of greater than 50 percent of normal output, pain in multiple joints (arthralgia), muscle pain (myalgia), frequent or recurring sore throat, or tender cervical or axillary lymph nodes, headaches of a new kind or greater severity, cognitive dysfunction (e.g. memory loss, impaired concentration); and the like. The presence of one or more of the above symptoms may be used to identify subjects suspected of having CFS.

Aspects of the invention further include methods of determining a treatment protocol/regimen for a subject suffering from CFS. The subject methods may be used to predict the risk of a CFS complication, where that result may be used to determine a treatment protocol. In other words, the subject methods may be used to predict the risk of a CFS subject developing a complication, the results of such a prediction being useful in determining a prophylactic treatment to prevent the complication. The subject methods may also be used to diagnose the cause of an existing complication, wherein that result may be used to determine a treatment for that complication. A complication is an unfavorable evolution of a disease, a health condition or a medical treatment. Complications could be a natural evolution of CFS, or a result of a medical treatment that produces adverse effects and/or produces new health problem(s) by itself, or a new disease that develops as a result of CFS. Indications of complications include but are not limited to indications that the CFS has become worse in its severity or shows an increased number of signs, symptoms or new pathologies.

In practicing methods according to certain embodiments, a subject or patient sample, e.g., a stool sample or intestinal biopsy, e.g., is assayed (i.e. tested or analyzed) to make the evaluation, e.g., to determine whether the host or subject from whom the assayed sample was obtained is susceptible to CFS, or to diagnose a subject as having CFS, or determine a treatment for a person suffering from CFS.

In certain embodiments, the sample obtained from the subject is assayed to obtain an intestinal bacteria assessment for the subject. Any convenient protocol for assaying a sample to obtain the intestinal bacterial assessment may be employed in the subject methods. In certain embodiments, a metagenomics protocol is employed. As used herein, the term “metagenomics” means the parallel analysis of nucleic acids recovered from multiple microorganisms (e.g., bacteria, archea, etc.) in a sample. Nucleic acids can be recovered by any method known in the art. In some embodiments, the nucleic acids are recovered together from the entire sample such that prior to analysis it is unknown from which microorganism any given nucleic acid molecule originated. In some embodiments, the sample contains an unknown mixture and/or quantity of microorganisms. In some embodiments, microorganisms present in the sample are isolated from one another by means known in the art (e.g., culture plating) prior to the extraction of nucleic acids such that it is known from which microorganism any given nucleic acid molecule originated.

Analysis of the recovered nucleic acids refers to sequencing the nucleic acids (which can be RNA or DNA) and subsequent evaluation of the sequences. Such evaluation can include the determination of the total number of different microorganisms present in the sample (in cases where this was unknown), the qualitative identification of the type (i.e., kingdom, phylum, class, order, family, genus, species, and/or sub-species) of microorganism(s) present in the sample (in cases where this was unknown), the quantification of the number of microorganisms of any particular type present in the sample, the identification of the relative number of different types of microorganisms present in the sample, the relatedness of the microorganisms present in the sample, etc. In some embodiments, the evaluation of nucleic acid sequences includes the derivation a diversity index. Diversity index is well known in the art, and in general is a quantitative measure of the diversity of a given sample. As used herein, the diversity index refers to the diversity of microorganisms present in the sample.

Sequencing of the nucleic acids recovered from the sample can be performed by any convenient method known in the art. For example, sequencing can be performed by Sanger sequencing, high-throughput sequencing (i.e. massive parallel sequencing, e.g., SOLiD sequencing by ligation, pyrosequencing, Illumina/Solexa reversible dye-terminator sequencing, Ion Torrent sequencing, single molecule real time sequencing, nanopore sequencing, etc.), etc.

In some embodiments, recovered nucleic acids are subjected to a selection step prior to analysis. A selection step can include the selective purification or amplification (e.g., by PCR) of a particular region or type of nucleic acid (e.g., PCR amplification of 16S rDNA, which is the region of genomic DNA within a genome that encodes 16S rRNA). For example, in some embodiments, the recovered nucleic acids are used as templates for a PCR reaction to amplify a specific region of DNA (e.g., 16S rDNA), which is then sequenced for subsequent evaluation.

In some embodiments, the recovered nucleic acids are sequenced prior to a selection step such that sequences are produced for all recovered nucleic acids. Subsequent evaluation steps can include all produced sequences, or can instead focus on a specific subset of nucleic acid sequences (e.g., 16S rDNA).

Metagenomic methods of interest are found in the scientific literature, for example: Li et al, Brief Bioinform. Jul 6, 2012: Ultrafast clustering algorithms for metagenomic sequence analysis (PMID: 22772836); Prakash et al., Brief Bioinform. Jul 6, 2012: Functional assignment of metagenomic data: challenges and applications (PMID: 22772835); Andreote et al. PLoS One. 2012;7(6):e38600: The microbiome of brazilian mangrove sediments as revealed by metagenomics; Wooley et al. PLoS Comput Biol. Feb 26, 2010;6(2):e1000667: A primer on metagenomics; etc. Metagenomic methods of interest are also found in the U.S. patent literature, for example: U.S. Pat. No. 7,749,366, U.S. Pat. No. 7,910,522, U.S. 20120129706, U.S. 20120129794, U.S. 20120004111, U.S. 20100279882, etc; the disclosures of which methods are herein incorporated by reference.

Following obtainment of the intestinal bacterial assessment from the sample being assayed, the assessment is evaluated to determine if the subject is susceptible/resistant to CFS, or to determine a treatment in a subject that is suffering from CFS. In certain embodiments, the obtained assessment may be compared with a reference or control to make a diagnosis regarding the CFS phenotype of the cell or tissue, and therefore host, from which the sample was obtained/derived. The terms “reference” and “control” as used herein mean a standardized intestinal bacterial assessment to be used to interpret the intestinal bacteria assessment of a given patient and assign a prognostic class thereto. The reference or control may be an intestinal bacteria assessment that is obtained from a sample of a subject known to have a particular phenotype, e.g., a susceptibility assessment, and therefore may be a positive reference or control assessment. Alternatively, the reference/control assessment may be from a sample from a subject known to not have the desired phenotype, and therefore be a negative reference/control assessment.

The specific bacteria of interest that are evaluated may vary, where species of interest include, but are not limited to: Prevotella ssp., Asaccharobacter ssp., Lactonifator spp., Eubacterium spp., Turicibacter ssp., Ruminococcus., Enterococcus ssp., Holdemania ssp., Roseburia ssp., Alistipes ssp. and Ethanoligenens ssp. and combinations thereof.

The intestinal bacterial assessment may also be a ratio of populations of bacterial groups, e.g., the ratio of Firmicutes/Bacteroidetes.

In certain embodiments, the above-obtained assessment is employed to determine if the subject is susceptible/resistant to CFS, or to diagnose a subject with CFS, or to predict if a subject suffering from CFS will develop a particular complication. In certain embodiments, the above-obtained information is employed to give a refined probability determination as to whether a subject is susceptible to

CFS, or to diagnose a subject with CFS, or to give a refined determination as to whether a subject suffering from CFS will develop a particular complication.

In some embodiments, determining that the subject is susceptible/resistant to CFS, or diagnosing a subject with CFS, or predicting if a subject suffering from CFS will develop a particular complication may be made by employing the results of the aforementioned analysis in conjunction with the identification of symptoms known in the art to be associated with CFS, e.g. a new onset (not lifelong) of unexplained, persistent fatigue unrelated to exertion and not substantially relieved by rest, impaired memory or concentration, post-exertional malaise, where physical or mental exertions bring on “extreme, prolonged exhaustion and sickness”, un-refreshing sleep, muscle pain (myalgia), pain in multiple joints (arthralgia), headaches of a new kind or greater severity, frequent or recurring sore throat, or tender cervical or axillary lymph nodes. In addition, the presence of symptoms that are attributable to other conditions, so as to exclude a determination of susceptibility to CFS or a CFS diagnosis, e.g. symptoms of mononucleosis, Lyme disease, lupus, multiple sclerosis, fibromyalgia, primary sleep disorders, severe obesity and major depressive disorders, may be employed.

In some embodiments, the above-obtained information is employed to determine a treatment for a subject suffering from CFS. Treatments of interest include, but are not limited to, antihistamines, prescription sleep medications, analgesics, anti-depressants, dietary supplements, alternative medicine, cognitive behavior therapy (a form of psychological therapy used to treat chronically ill subjects), graded exercise therapy (a form of physical therapy), and pacing (an energy management strategy in which subjects are advised to set manageable daily activity/exercise goals and balance activity and rest to avoid over-exertion which may worsen symptoms). Classes of therapeutic treatments of interest include, but are not limited to, therapeutic antibodies, peptide mimetics, small molecule agonists or antagonists, siRNA-based therapeutics, and gene therapy.

In some embodiments, providing an evaluation of a subject for CFS, e.g. a prediction of a subject's susceptibility to developing CFS , a diagnosis of CFS in a subject suspected of having CFS, and/or a determination of a treatment regimen for a CFS subject, includes generating a written report that includes an assessment of the subject's susceptibility to developing CFS, i.e. a “susceptibility assessment”, the subject's current state of health i.e. a “CFS diagnosis assessment”, and/or possible treatment regimens, i.e. a “treatment assessment”. Thus, a subject method may further include a step of generating or outputting a report providing the results of a susceptibility assessment, a CFS diagnosis assessment, or treatment assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a susceptibility assessment, a CFS diagnosis assessment, or treatment assessment and its results. A subject report can be completely or partially electronically generated. A subject report includes at least a susceptibility prediction, i.e. a prediction as to the susceptibility of a subject to developing CFS; or a CFS diagnosis, i.e. whether a subject does or does not have CFS; or a suggested treatment regimen, i.e. a course of treatment to be followed. A subject report can further include one or more of: 1) information regarding the testing facility; 2) service provider information; 3) subject data; 4) sample data; 5) an assessment report, which can include various information including: a) test data, where test data can include a bacterial assessment; 6) other features.

The report may include information about the testing facility, which information is relevant to the hospital, clinic, or laboratory in which sample gathering and/or data generation was conducted. Sample gathering can include obtaining a fluid sample, e.g. blood, saliva, urine etc.; or a tissue sample, e.g. a tissue biopsy, a swab of the lining of the mouth or nose, a collect hair follicle, etc. from a subject. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the susceptibility prediction, CFS diagnosis, or proposed treatment regimen) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed in the susceptibility prediction, such as the source of biological sample obtained from the subject (e.g. blood, saliva, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prediction of the likelihood that the patient will develop CFS. The assessment portion of the report can optionally also include a Recommendation(s). For example, where the results indicate an increased susceptibility to developing CFS, the recommendation can include a recommendation that diet or lifestyle be altered or medical intervention be provided as recommended in the art.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., susceptibility prediction).

### Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of identifying the presence of the target polymorphisms, where such reagents may include nucleic acid primers, arrays of nucleic acid probes, antibodies to polymorphic polypeptides (e.g., immobilized on a substrate), signal producing system reagents, etc., depending on the particular detection protocol to be performed. For example, reagents may include universal 16 s PCR primers, as described above.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### Computer Systems

Also of interest are computer systems configured to perform one or more aspects of the methods, i.e., to evaluate a subject for a chronic immune disease according to methods described herein. As would be recognized by one of skilled in the art, many different hardware options and data structures can be employed to implement the method described below.

Substantially any general-purpose computer can be configured to a functional arrangement for the methods and programs disclosed herein. The hardware architecture of such a computer is well known by a person skilled in the art, and can comprise hardware components including one or more processors (CPU), a random-access memory (RAM), a read-only memory (ROM), an internal or external data storage medium (e.g., hard disk drive). A computer system can also comprise one or more graphic boards for processing and outputting graphical information to display means. The above components can be suitably interconnected via a bus inside the computer. The computer can further comprise suitable interfaces for communicating with general-purpose external components such as a monitor, keyboard, mouse, network, etc. In some embodiments, the computer can be capable of parallel processing or can be part of a network configured for parallel or distributive computing to increase the processing power for the present methods and programs. In some embodiments, the program code read out from the storage medium can be written into a memory provided in an expanded board inserted in the computer, or an expanded unit connected to the computer, and a CPU or the like provided in the expanded board or expanded unit can actually perform a part or all of the operations according to the instructions of the program code, so as to accomplish the functions described below. In other embodiments, the method can be performed using a cloud computing system. In these embodiments, the datafiles and the programming can be exported to a cloud computer, which runs the program, and returns an output to the user.

The memory of a computer system can be any device that can store information for retrieval by a processor, and can include magnetic or optical devices, or solid state memory devices (such as volatile or non-volatile RAM). A memory or memory unit can have more than one physical memory device of the same or different types (for example, a memory can have multiple memory devices such as multiple drives, cards, or multiple solid state memory devices or some combination of the same). With respect to computer readable media, “permanent memory” refers to memory that is permanent. Permanent memory is not erased by termination of the electrical supply to a computer or processor. Computer hard-drive ROM (i.e., ROM not used as virtual memory), CD-ROM, floppy disk and DVD are all examples of permanent memory. Random Access Memory (RAM) is an example of non-permanent (i.e., volatile) memory. A file in permanent memory can be editable and re-writable.

Operation of computer is controlled primarily by an operating system, which is executed by a central processing unit. The operating system can be stored in system memory, as desired. In some embodiments, the operating system 4 includes a file system. In addition to operating system, one possible implementation of system memory includes a variety programming files and data files for implementing the methods of the invention, e.g., as described above, where these implementation files are collectively referred to herein as an evaluation module, where the evaluation module is configured to perform the methods, e.g., as described herein.

In certain embodiments, instructions in accordance with the method described herein can be coded onto a computer-readable medium in the form of “programming”, where the term “computer readable medium” as used herein refers to any storage or transmission medium that participates in providing instructions and/or data to a computer for execution and/or processing. Examples of storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer.

The computer-implemented method described herein can be executed using programming that can be written in one or more of any number of computer programming languages. Such languages include, for example, Java (Sun Microsystems, Inc., Santa Clara, Calif.), Visual Basic (Microsoft Corp., Redmond, Wash.), and C++ (AT&T Corp., Bedminster, N.J.), as well as any many others.

### Therapeutic Methods and Compositions

As summarized above, aspects of the invention include methods for treating a host suffering from a chronic immune disease, e.g., MS or CFS. In practicing the subject methods, an effective amount of an intestinal flora modulatory agent is administered to the host suffering from the chronic immune disease. The intestinal flora modulatory agent may be any convenient agent, such as: antibiotics, prebiotics and probiotics and combinations thereof.

In the subject methods, the active agent(s) may be administered to the host using any convenient means capable of resulting in the desired treatment. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intracheal,etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. antisense composition, it may be introduced into tissues or host cells by any number of routes, including viral infection, microinjection, or fusion of vesicles. Jet injection may also be used for intramuscular administration, as described by Furth et al., supra. The agent may be coated onto gold microparticles, and delivered intradermally by a particle bombardment device, or “gene gun” as described in the literature as described by Tang et al., supra.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

As mentioned above, by treatment is meant that at least an amelioration of the symptoms associated with the chronic immune disease, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the chronic immune disease condition.

In treating subjects according to the subject invention, the active agent may be administered by itself, or in conjunction with one or more additional chronic immune disease therapeutic agents. For example, the intestinal flora modulatory agent may be administered in conjunction with an interferon agent, e.g., interferon or an inducer thereof, as described above and in U.S. Pat. No. 6,013,253, the disclosure of which is herein incorporated by reference.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The following examples are offered by way of illustration and not by way of limitation.

### EXPERIMENTAL

### Introduction

We used high-throughput 16s rDNA sequencing to investigate the presence of specific alterations in the gut flora of ME-CFS patients from Belgium and Norway.

### Example I.

### Methods

39 ME-CFS patients and 35 healthy controls were included in the study. Bacterial DNA was extracted from stabilized stool samples and PCR amplification was performed on conserved 16S rDNA regions. PCR amplicons were then sequenced using Roche FLX 454 genome sequencer (6000-10000 sequences per sample). Bacteria were classified by phylum, family and genus; diversity indexes (Chao and Shannon) were also calculated. Data were analyzed using Mann-Whitney test and step-wise linear discriminant analysis.

### Results

ME-CFS patients presented altered levels of specific bacterial populations: Prevotella, Asaccharobacter, Lactonifactor, Eubacterium. Linear discriminant analysis showed that a significant (p<0,001) discrimination between control and patient populations could be achieved by using a combination of Asaccharobacter, Turicibacter, Ruminococcus and Enterococcus as variables. Differences could be seen between males and females, as well as between people from different geographical origins (Belgium vs. Norway).

### Conclusions

ME-CFS patients present significant alterations of their gut flora composition. Metagenomics is a useful tool to diagnose dysbiosis in ME-CFS patients and to help design treatments based on gut flora modulation (antibiotics, pre- and probiotics supplementation).

### Example II.

43 ME-CFS patients and 36 healthy controls were included in the study. Bacterial DNA was extracted from stool samples, PCR amplification was performed on conserved 16S rDNA regions, and PCR amplicons were sequenced using Roche FLX 454 sequencer. Bacteria were classified by phylum, family and genus. Data were analyzed using Mann-Whitney test and linear discriminant analysis.

The composition of the gut microflora was found to differ between Belgian controls and Norwegian controls: Norwegians showed higher percentages of specific Firmicutes populations (Roseburia, Holdemania) and lower proportions of most Bacteroidetes genera. These differences may be due to genetic and/or diet factors. A highly significant separation (sign. <0,001) could be achieved between Norwegian controls and Norwegian patients: patients presented increased proportions of Lactonifactor and Alistipes, as well as a decrease in several Firmicutes and Actinobacteria populations. In Belgian subjects the patient/control separation was less pronounced, however some abnormalities observed in Norwegian patients were also found in Belgian patients (altered proportions of Lactonifactor, Ethanoligenens, Ruminococcus and Eubacterium).

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of evaluating a subject for Chronic Fatigue Syndrome (CFS), the method comprising:
(a) obtaining an intestinal bacteria assessment for the subject; and
(b) employing the assessment to provide an evaluation of a subject for CFS.

2. The method according to claim 1, wherein the intestinal bacterial assessment comprises a comparison of a measured population of one or more intestinal bacterial species to a control.

3. The method according to claim 2, wherein the one or more intestinal bacterial species is selected from the group consisting of Prevotella ssp., Asaccharobacter ssp., Lactonifator spp., Eubacterium spp., Turicibacter ssp., Ruminococcus., Enterococcus ssp., Holdemania ssp., Roseburia ssp., Alistipes ssp. and Ethanoligenens ssp. and combinations thereof.

4. The method according to claim 3, wherein the assessment comprises a comparison for two or more species selected from the group consisting of Prevotella ssp., Asaccharobacter ssp., Lactonifator spp., Eubacterium spp., Turicibacter ssp., Ruminococcus., Enterococcus ssp., Holdemania ssp., Roseburia ssp., Alistipes ssp. and Ethanoligenens ssp.

5. The method according to claim 1, wherein the assessment is obtained using a 16s rRNA protocol.

6. The method according to claim 1, wherein the intestinal bacterial assessment comprises a ratio of measured populations of two bacterial groups.

7. The method according to claim 6, wherein the two bacterial groups are Firmicutes and Bacteroidetes.

8. The method according to claim 1, wherein the assessment is obtained using a metagenomics protocol.

9. The method according to claim 8, wherein the metagenomics protocol produces 16s rRNA amplicons.

10. The method according to claim 1, wherein the assessment comprises a diversity index.

11. The method according to claim 1, wherein the method comprises obtaining a sample from the subject and assaying the sample to obtain the assessment.

12. The method according to claim 11, wherein the sample is a stool sample.

13. The method according to claim 11, wherein the sample is an intestinal biopsy.

14. The method according to claim 1, wherein the evaluation is a prediction of whether a subject is susceptible to Chronic Fatigue Syndrome.

15. The method according to claim 1, wherein the evaluation is a diagnosis of CFS in a subject suspected of having CFS.

16. The method according to claim 1, wherein the evaluation is a determination of a treatment for a subject with CFS.

17. A kit for evaluating a subject for Chronic Fatigue Syndrome (CFS), the kit comprising:
a) universal 16S rRNA primers; and
b) control information for employing an assessment of intestinal bacteria in a subject to provide an evaluation of a subject for CFS.

18. The kit according to claim 17, wherein the kit further comprises one or more PCR reagents.

19. A method for treating a subject for a chronic immune disease, the method comprising administering to the subject an effective amount of an intestinal flora modulatory agent to treat the subject for the chronic immune disease.

20. The method according to claim 19, wherein the chronic immune disease is selected from the group consisting of CFS and MS.

21. The method according to claim 20, wherein the intestinal flora modulatory agent is selected from the group consisting of: antibiotics, prebiotics and probiotics and combinations thereof.

22. The method according to claim 19, wherein the subject is a mammal.

23. The method according to claim 22, wherein the mammal is a human.

24. The method according to claim 19, wherein the subject is diagnosed as having the chronic immune disease prior to the administering step.
